(19) United States
(12) Patent Application Publication
(10) Pub. No.: US 2009/0204083 A1
(43) Pub. Date: Aug. 13, 2009

(54) Method and Apparatus for Treating Stenoses at Bifurcated Regions

(75) Inventors: Sinead O'Donnell, Ballybrit (IE); Noreen Moloney, Ballybrit (IE); Colm McCormack, Kilcock (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/030,693

(22) Filed: Feb. 13, 2008

(51) Int. Cl. A61M 25/00; A61M 31/00; A61B 6/00

(52) U.S. Cl. 604/284; 604/523; 604/509; 600/434

(57) ABSTRACT

A bifurcated catheter includes a first catheter branch having a first distal portion and a second catheter branch having a second distal portion. The first and second distal portions are linked together for delivery to the bifurcated region. Upon delivery to the bifurcated region, the first and second distal portions are released from each other such that the first and second catheter branches may be tracked into first and second vessel branches, respectively. The first and second distal portions can be linked together by inserted the second distal portion into a skive in the first distal portion, adhesive beat bonding the distal portions together, a coil wrapped around the distal portions, or a spiral cut in the first distal portion sized and shaped to received a guidewire.

### FIELD OF THE INVENTION

The invention relates generally dilatation catheters, stents and grafts for dilating strictures or stenoses in the human body. More particularly, the invention relates to a balloon catheter, including a delivery system for a bifurcated endoluminal prosthesis, for treating site or sites at or near a bifurcation of a body lumen.

### BACKGROUND OF THE INVENTION

The use of balloon catheters with or without stents to treat strictures, stenoses, or narrowings in various parts of the human body is well known in the prior art. Devices of numerous designs have been utilized for angioplasty, stents and grafts or combination stent/grafts. Varied catheter designs have been developed for the dilatation of stenoses and to deliver prostheses to treatment sites within the body lumen.

Devices developed specifically to address the problems that arise in the treatment of stenoses at or near the site of a bifurcation of a body lumen are known in the art. Examples of catheters for use in treating bifurcated lumens or delivery systems for bifurcated endoluminal prostheses are shown in U.S. Pat. No. 5,720,735 to Dorros, U.S. Pat. No. 5,669,924 to Shaknovich, U.S. Pat. No. 5,749,825 to Fischell, et al., and U.S. Pat. No. 5,718,724 to Goicoechea et al.

Various techniques have been used to deliver multiple prostheses in order to provide radial support to both a main blood vessel, for example, and contemporaneously to side branches of the blood vessel. Further, single bifurcated stents and grafts have been developed in order to treat such conditions at the site of a branch of a body lumen. A bifurcated stent and/or graft typically comprises a tubular body or trunk and two tubular legs. Examples of bifurcated stents are shown in U.S. Pat. No. 5,723,004 to Dereume et al., U.S. Pat. No. 4,994,071 to MacGregor, and European Pat. Application EP 0 804 907 A2 to Richter, et al.

Illustrative procedures involving balloon catheters include percutaneous transluminal angioplasy (PTA) and percutaneous transluminal coronary angioplasty (PTCA), which may be used to reduce arterial build-up such as caused by the accumulation of atherosclerotic plaque. These procedures involve passing a balloon catheter over a guide wire to a stenosis with the aid of a guide catheter. The guide wire extends from a remote incision to the site of the stenosis, and typically across the lesion. The balloon catheter is passed over the guide wire, and ultimately positioned across the lesion.

Once the balloon catheter is positioned appropriately across the lesion, (e.g., under fluoroscopic guidance), the balloon is inflated, which breaks the plaque of the stenosis and causes the arterial cross section to increase. Then the balloon is deflated and withdrawn over the guide wire into the guide catheter, and from the body of the patient.

In many cases, a stent or other prosthesis must be implanted to provide permanent support for the artery. When such a device is to be implanted, a balloon catheter which carries a stent on its balloon is deployed at the site of the stenosis. The balloon and accompanying prosthesis are positioned at the location of the stenosis, and the balloon is inflated to circumferentially expand and thereby implant the prosthesis. Thereafter, the balloon is deflated and the catheter and the guide wire are withdrawn from the patient.

Administering PTCA and/or implanting a stent at a bifurcation in a body lumen poses further challenges for the effective treatment of stenoses in the lumen. For example, dilating a vessel at a bifurcation may cause narrowing of an adjacent branch of the vessel. In response to such a challenge, attempts to simultaneously dilate both branches of the bifurcated vessel have been pursued. These attempts include deploying more than one balloon, more than one prosthesis, a bifurcated prosthesis, or some combination of the foregoing.

However, simultaneously deploying multiple and/or bifurcated balloons with or without endoluminal prostheses, hereinafter individually and collectively referred to as a bifurcated assembly, requires highly accurate placement of the assembly. Specifically, deploying a bifurcated assembly requires positioning a main body of the assembly within the trunk of the vessel adjacent the bifurcation, and then positioning the independent legs of the assembly into separately branching legs of the body lumen.

Tracking a bifurcated assembly to a treatment site also presents additional challenges to the more standard PTCA procedure. For example, a bifurcated catheter must be tracked to the site as a unitary device until it reaches the bifurcation. Once it reaches the bifurcated treatment site, it must be positioned within the separate branches of the vessel. Therefore, it must be a unitary device during tracking and be a bifurcated device for treatment.

In order to achieve the foregoing objectives, objectives, two guide wires are typically required, one for placement of the assembly into each branch of the bifurcated vessel. Devices known in the prior art fail to track and position a device requiring two guide wires in an expeditious fashion by failing to prevent the entanglement of the wires or other complications which would prevent proper placement of the assembly and/or a smooth withdrawal the catheter and of the guide wires.

Further, devices known in the prior art fail to provide a bifurcated assembly, the distal portion of which functions as a unitary device during tracking and as a bifurcated device for positioning and deployment.

In view of the foregoing, it is an object of this invention to provide improved catheters and methods for use with multiple guide wires for delivering balloon catheters and prostheses designed to treat stenoses at or near a bifurcation of a body lumen.

### BRIEF SUMMARY OF THE INVENTION

The present disclosure relates to bifurcated catheters which can be linked together such that they can be tracked to a bifurcated region of a body lumen over a single guide wire. A bifurcated catheter according to the present disclosure includes a first catheter branch having a first distal portion and a second catheter branch having a second distal portion. The first and second distal portions are linked together for delivery to the bifurcated region. Upon delivery to the bifurcated region, the first and second distal portions are released from each other such that the first and second catheter branches may be tracked into first and second vessel branches, respectively.

In an embodiment, the first and catheter branches are linked together by inserting the distal portion of the second catheter branch into a skive in the distal portion of the first catheter branch. Upon delivery to the bifurcated region, a force separates the second distal portion from the skive such that the first and second catheter branches may be advanced separately into the first and second vessel branches.

In another embodiment, the first and second distal portions are adhesively or heat bonded together. The bifurcated catheter is advanced over a singe guide wire to the bifurcation region. Upon reaching the bifurcated region, a second guide wire is advanced through the second catheter branch into the second branch vessel. The first and second catheter branches are then advanced over the first and second guide wires, respectively, and the force exerted at the carina of the bifurcation is sufficient to break the bond.

In another embodiment, a coil is wrapped around the first and second distal portions to couple them together. A pull string is coupled to the coil. The first and second catheter branches are coupled together for delivery to the bifurcation region. Upon reaching the bifurcation region, the pull string is pulled, causing the coil to unwind and release the first and second catheter branches from each other. The first and second catheter branches can then be advanced into their respective branch vessels.

In another embodiment, the first distal portion includes a spiral cut sized and shaped to receive a guide wire. A first guide wire is advanced to the bifurcated region. A proximal portion of the first guide wire is inserted into a first distal opening of the first distal portion, through the spiral cut, and into a second distal opening of the second distal portion. The bifurcated catheter is advanced to the bifurcated region over the first guide wire. A second guide wire is advanced through the first catheter branch and into a first branch vessel. The first guide wire is pulled back slightly and directed to a second branch vessel. As the first guide wire is advanced into the second branch vessel, the first guide wire pulls through the spiral cut, thereby releasing the first and second catheter branches from each other. The first and second catheter branches are then advanced into their respective branch vessels.

Further features of the invention, its nature and various advantages, will be more apparent from the accompanying drawings and the following detailed description of the preferred embodiments.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a simplified, partial, elevational view of a bifurcated catheter in accordance with an embodiment of the present invention.

FIG. 2 illustrates a partial, elevational view of the distal portion of a first catheter branch of the catheter of FIG. 1.

FIG. 3 illustrates a partial, elevational view of the distal portion of the catheter of FIG. 1.

FIG. 3A illustrates a partial, elevational view of the distal portion of the catheter of FIG. 1 including perfusion holes for separation of the distal portions of the first and second catheter branches.

FIG. 3B illustrates a partial, elevational view of the distal portion of the catheter of FIG. 1 including a separation balloon for separation of the distal portions of the first and second catheter branches.

FIG. 4 illustrates a partial, elevational view of a distal portion of the catheter of FIG. 1 with the first and second catheter branches separated.

FIG. 4A illustrates a partial, elevational view of a distal portion of the catheter of FIG. 3A with the first and second catheter branches separated using perfusion holes.

FIG. 4B illustrates a partial, elevational view of a distal portion of the catheter of FIG. 3B with the separation balloon inflated to separate the first and second catheter branches.

FIG. 5 illustrates the catheter of FIG. 1 in vivo, following the step of threading the catheter over a guide wire.

FIG. 6 illustrates the catheter of FIG. 1 in vivo when the catheter has been delivered to the bifurcation site.

FIG. 7 illustrates the catheter of FIG. 1 after a distal portion of the second catheter branch has been removed from the skive in the distal portion of the first catheter branch.

FIG. 8 illustrates the catheter FIG. 1 after the catheter branches have been advanced into the respective vessel branches.

FIG. 9 illustrates the catheter of FIG. 1 subsequent to inflation of the balloon(s).

FIG. 10 illustrates a simplified, partial, elevational view of a bifurcated catheter in accordance with another embodiment of the present invention.

FIG. 11 illustrates a partial, elevational view of the distal portion of the catheter of FIG. 10.

FIG. 12 illustrates the catheter of FIG. 10 in vivo, following the step of threading the catheter over a guide wire.

FIG. 13 illustrates the catheter of FIG. 10 after a second guide wire has been threaded through the second guide wire lumen and into the second branch vessel.

FIG. 14 illustrates the catheter of FIG. 10 as it approaches the bifurcation and the distal portions of the first and second catheter branches begin to separate.

FIG. 15 illustrates the catheter FIG. 10 after the catheter branches have been advanced into the respective vessel branches.

FIG. 16 illustrates the catheter of FIG. 10 subsequent to inflation of the balloon(s).

FIG. 17 illustrates a simplified, partial, elevational view of a bifurcated catheter in accordance with another embodiment of the present invention.

FIG. 18 illustrates a partial, elevational view of the distal portion of the catheter of FIG. 17.

FIG. 19 illustrates the catheter of FIG. 17 in vivo, following the step of threading the catheter over a guide wire.

FIG. 20 illustrates the catheter of FIG. 17 after it has been advanced to the bifurcation site.

FIG. 21 illustrates the catheter of FIG. 17 after a second guide wire has been advanced through the guide wire lumen of the first catheter branch and into the first branch vessel.

FIG. 22 illustrates the catheter FIG. 17 after the first guide wire has been retracted and is being directed to the second branch vessel.

FIG. 23 illustrates the catheter of FIG. 17 as the first guide wire is advanced into the second branch vessel, thereby being pulled through the spiral of the distal portion of the first catheter branch.

FIG. 24 illustrates the catheter of FIG. 17 after the catheter branches have been advanced into the respective vessel branches.

FIG. 25 illustrates the catheter of FIG. 17 subsequent to inflation of the balloon(s).

FIG. 26 illustrates a simplified, partial, elevational view of a bifurcated catheter in accordance with another embodiment of the present invention.

FIG. 27 illustrates a partial, elevational view of the distal portion of the catheter of FIG. 26.

FIG. 28 illustrates the catheter of FIG. 26 in vivo, following the step of threading the catheter over a guide wire.

FIG. 29 illustrates the catheter of FIG. 26 after a second guide wire has been advanced through the second catheter branch and into the second branch vessel.

FIG. 30 illustrates the catheter of FIG. 26 as the pull wire is being pulled to uncouple the distal portions of the first and second catheter branches.

FIG. 31 illustrates the catheter of FIG. 26 after the catheter branches have been advanced into the respective vessel branches.

FIG. 32 illustrates the catheter of FIG. 26 subsequent to inflation of the balloon(s).

FIG. 33 illustrates the distal portion of another embodiment of a bifurcated catheter.

FIG. 34 illustrates a distal end view of a manufacturing step of the catheter of FIG. 33.

### DETAILED DESCRIPTION OF THE INVENTION

Specific embodiments of the present disclosure are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

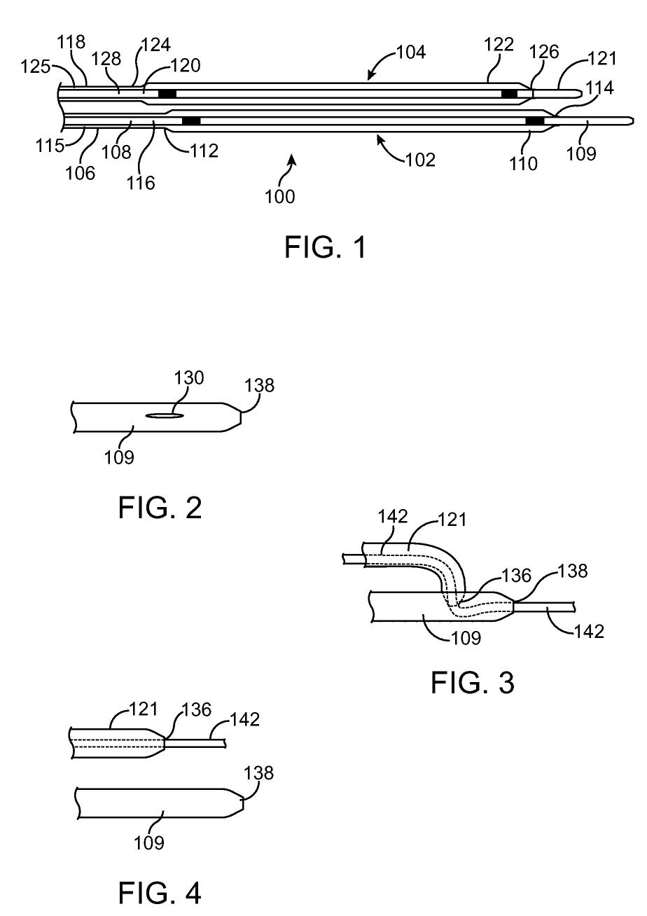

An illustrative embodiment of a catheter 100 constructed in accordance with this disclosure is shown in FIG. 1. The proximal portion of catheter 100 is towards the left in FIG. 1, and the distal portion is towards the right. Catheter 100 may comprise two separate tubular structures linked at particular points along their lengths, or it may consist of a single tubular structure with multiple lumens in its interior.

FIG. 1 depicts a catheter having two branches and two balloons, but more than two balloons may be utilized with the present invention. Alternatively, a bifurcated balloon, either alone or in combination with one or more standard balloons may be utilized.

Catheter 100 includes a first catheter branch 102 and a second catheter branch 104. First catheter branch 102 includes a first outer shaft 106, a first inner shaft 108, and a first balloon 110. A proximal end of first balloon 110 is mounted to a distal portion of first outer shaft 106 at a first proximal junction 112. A distal end of first balloon 110 is mounted to a distal portion of first inner shaft 108 at a first distal junction 114. A first inflation lumen 115 extends between first outer shaft 106 and first inner shaft 108, and is in communication with an interior of first balloon 110. A first guide wire lumen 116 extends through first inner shaft 108.

Similarly, second catheter branch 104 includes a second outer shaft 118, a second inner shaft 120, and a second balloon 122. A proximal end of second balloon 122 is mounted to a distal portion of second outer shaft 118 at a second proximal junction 124. A distal end of second balloon 122 is mounted to a distal portion of second inner shaft 120 at a second distal junction 126. A second inflation lumen 125 extends between second outer shaft 118 and second inner shaft 120, and is in communication with an interior of second balloon 122. A first guide wire lumen 128 extends through second inner shaft 120.

First and second inflation lumens 115, 125 can be conventional, and extend from a proximal portion of catheter 100 outside the patient, which is not pictured. First and second inflation lumens 115, 125 are in fluid communication with the interiors of first balloon 110 and second balloon 122. Thus, first and second inflation lumens 115, 125 are used to supply pressurized inflation fluid to first balloon 110 and second balloon 122 when it is desired to inflate the balloons. Inflation lumens 115, 125 are also used to drain inflation fluid from first balloon 110 and second balloon 122 when it is desired to deflate the balloons.

Although first and second guide wire lumens 116, 128 are shown passing through the interior of first and second balloons 110, 122, they need not. For example, the lumens may be affixed to the exterior of the balloon, or the balloon may be formed with a plurality of folds through which the guide wire passes. Alternatively, the guide wire may pass through the folds of the balloon, as illustrated in U.S. Pat. No. 6,071,285 for a Rapid Exchange Folded Balloon Catheter and Stent Delivery System, the entirety of which is incorporated by reference herein. First and second guide wire lumens 116, 128 are distinct from first and second inflation lumens 115, 125 and are not in fluid communication with the interior of first and second balloons 110, 122. Further, first and second guide wire lumens 116, 128 can begin and terminate generally at any point along first and second catheter branches 102, 104, but preferably they extend distally of first and second balloons 110, 122, respectively.

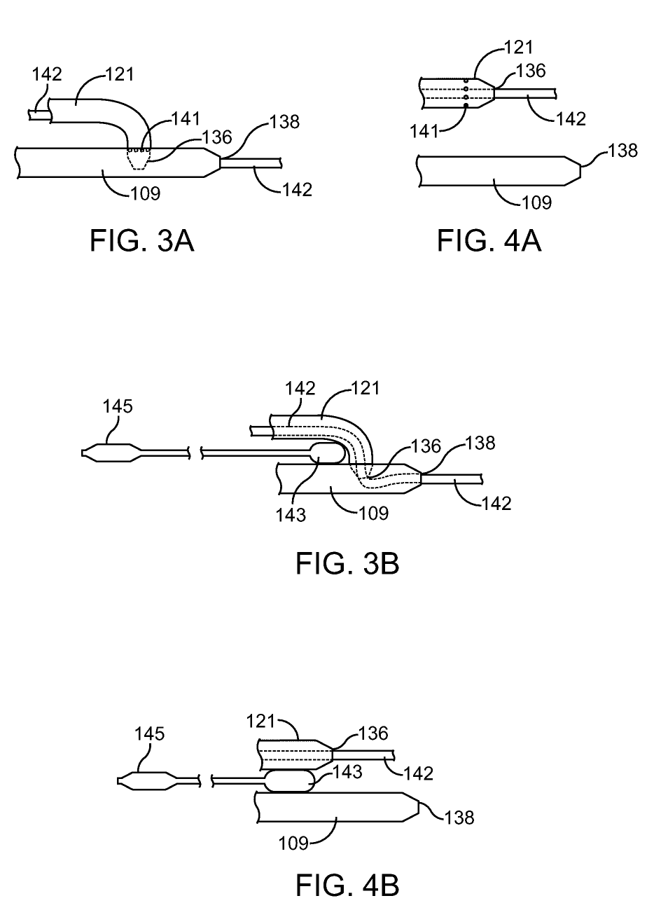

First catheter branch 104 further includes a skive or slit 130 in a distal portion 109 of second inner shaft 108, as shown in FIG. 2. Skive or slit 130 is sized and shaped such that it can receive a distal portion 121 of second inner shaft 121 therein, as shown in FIG. 3. By inserting distal portion 121 of second inner shaft 120 is disposed into skive or slit 130, first catheter branch 102 and second catheter branch 104 are coupled together for delivery to the site of a lesion. Once at the lesion site, a force separates distal portion 121 from skive or slit 130, thereby freeing second catheter branch 104 from first catheter branch 102, as shown in FIG. 4.

The force that separates second catheter branch 104 from skive or slit 130 can be provided through many different mechanisms, as would be understood by those of ordinary skill in the art. For example, and not by way of limitation, perfusion holes may be provided in the distal portion of one of the inner shafts. FIGS. 3A and 4A illustrate a distal portion of the catheter of FIG. 1 with perfusion holes 141 in distal portion 121 of second inner shaft 120. Perfusion holes 141 align with the junction of distal portion 121 and distal portion 109 when distal portion 121 is inserted into skive 120. Upon delivery of the catheter to the bifurcation site, saline or another suitable fluid is injected through second guide wire lumen 128, thereby causing distal portion 121 to separate from distal portion 109.

In another example shown in FIGS. 3B and 4B, a separation balloon 143 is positioned between distal portions 121 and 109. A lumen 147 runs between first catheter branch 102 and second catheter branch 104 to a proximally located inflation luer 145. An inflation fluid is injection through luer 145, lumen 147, and into balloon 143. Balloon 143 inflates, thereby separating distal portions 121 and 109, as shown in FIG. 4B. Balloon 143 may be an ultra low profile, low pressure balloon made from, for example latex or another material that expands quickly at low pressure and deflates quickly into its original shape. A no-fold balloon may be used as separation balloon 143 to maintain a low profile of the overall device. Upon separation of distal portions 121 and 109, balloon 143 may be withdrawn from the vessel.

In another example, a pull wire may be coupled to distal portion 121 of second inner shaft 120. After catheter 100 has been advanced to the bifurcation site, the pull wire is pulled to remove distal portion 121 from skive 130.

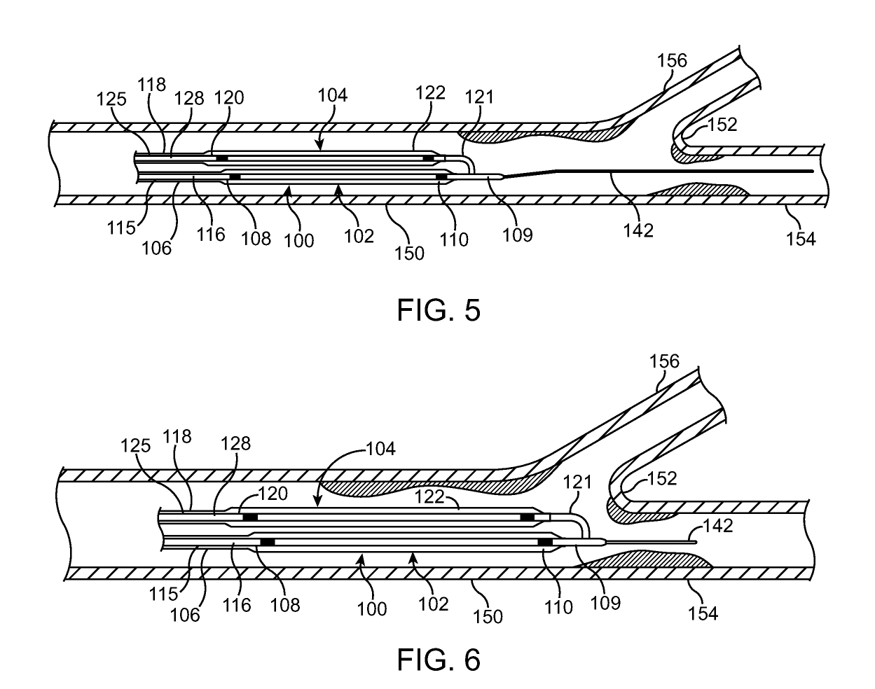

With reference to FIGS. 5-9, the manner of practicing the invention will now be discussed. A first guide wire 142 is in place in the body lumen. A proximal end of first guide wire 142 is threaded into a distal opening 138 of distal portion 109 of first inner shaft 108, into a distal opening 136 of distal portion 121 of second inner shaft 120 which is disposed within skive or slit 130. Second catheter branch 104 is threaded over first guide wire 142 while distal portion 121 of second inner shaft 120 is disposed within skive/slit 130, thereby coupling the distal portions of first and second catheter branches 102, 104 together, as shown in FIG. 5.

Catheter 100 is thus threaded over first guide wire 142 and tracked to a position at or near a bifurcation 152 of a vessel 150, as depicted in FIG. 6. A second guide wire 140 may be pre-installed through first guide wire lumen 116 such that second guide wire 140 is advanced with catheter 100 as catheter 100 is advanced to the bifurcation site 152. Alternatively, second guide wire 140 may be inserted in first guide wire lumen 116 after catheter 100 has reached the bifurcation site 152.

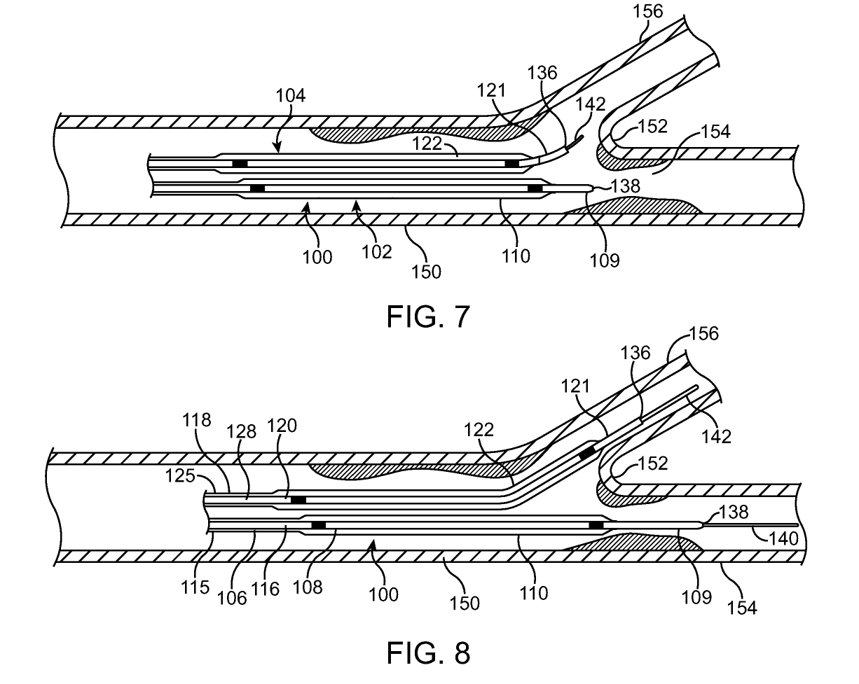

With catheter 100 positioned near bifurcation 152, distal portion 121 of second inner shaft 120 is removed from skive/slit 130, as shown in FIG. 7. As noted above, distal portion 121 of second inner shaft 120 may be removed from skive/slit 130 by saline infusion, a pull wire, or a separate balloon, as generally described above, or by other means as would be apparent to those of ordinary skill in the art.

With catheter 100 positioned near bifurcation 152 and distal portion 121 of second inner shaft 120 dislodged from skive/slit 130, first and second branches 102, 104 can then be positioned independently of one another such that first and second balloons 110, 122 may be positioned independently of each other. As shown in FIG. 8, second guide wire 140 is extended through distal opening 138 of distal portion 109 of first inner shaft 120. Second guide wire 140 is then extended into a first branch 154 of vessel 150 and first guide wire 142 is extended into second branch 156 of vessel 150.

With first guide wire 142 positioned within second branch 156 of vessel 150 and second guide wire 140 positioned within first branch 154 of vessel 150, catheter 100 may be further advanced such that first catheter branch 102 is disposed within first branch vessel 154 and second catheter branch is disposed within second branch vessel 156, as illustrated in FIG. 8.

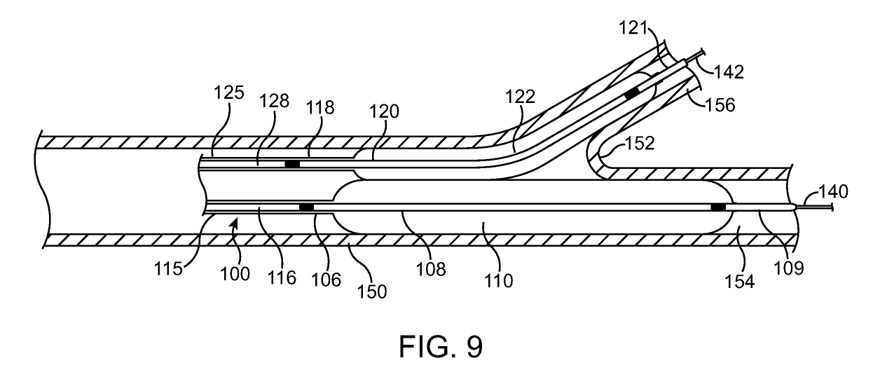

Once the entire assembly is properly positioned, pressurized fluid is supplied to first and second balloons 110, 122 through first and second inflation lumens 115, 125, as illustrated in FIG. 9. After first balloon 110 and second balloon 122 have been inflated as described above, first balloon 110 and second balloon 122 are deflated by draining the inflation fluid via first and second inflation lumens 115, 125. This allows the balloons to collapse in preparation for withdrawal of the assembly from vessel 150.

As would be understood by those of ordinary skill in the art, a bifurcated stent may be mounted on first and second balloons 110, 122 of catheter 100, as shown in FIGS. 2-2F of U.S. Pat. No. 6,129,738, the entirety of which is incorporated by reference herein. As noted in the '738 patent, a single bifurcated stent or but multiple stents, in place of or in combination with a bifurcated stent, may be deployed utilizing a bifurcated catheter of the present invention.

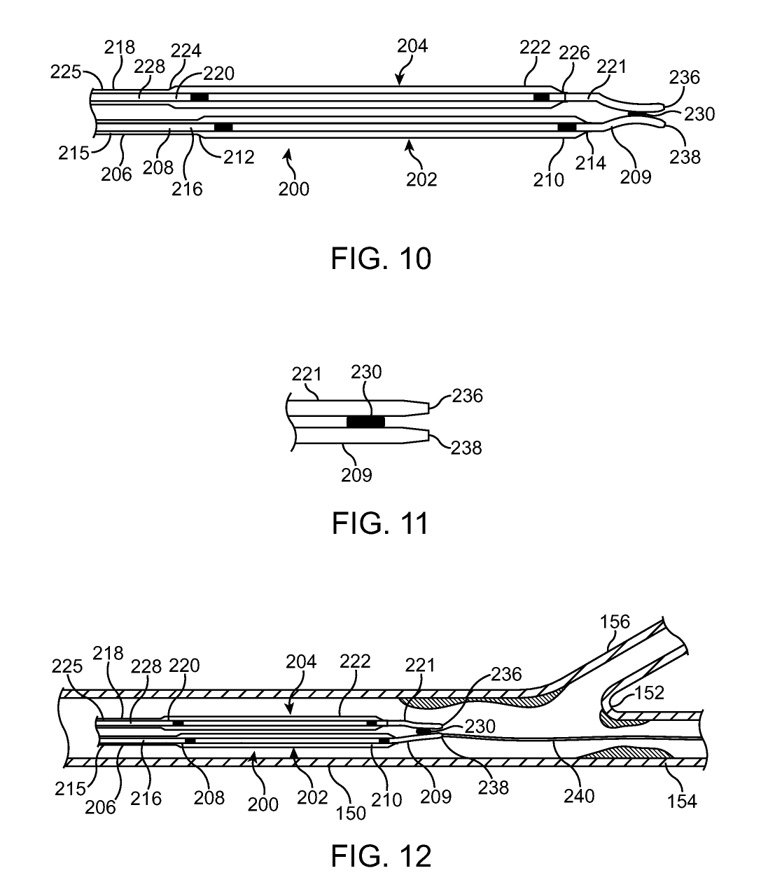

FIGS. 10-16 depict another embodiment of a catheter 200 in accordance with the present invention. Catheter 200 includes a first catheter branch 202 and a second catheter branch 204. First catheter branch 202 includes a first outer shaft 206, a first inner shaft 208, and a first balloon 210. A proximal end of first balloon 210 is mounted to a distal portion of first outer shaft 206 at a first proximal junction 212. A distal end of first balloon 210 is mounted to a distal portion of first inner shaft 108 at a first distal junction 214. A first inflation lumen 215 extends between first outer shaft 206 and first inner shaft 208, and is in communication with an interior of first balloon 210. A first guide wire lumen 216 extends through first inner shaft 208.

Similarly, second catheter branch 204 includes a second outer shaft 218, a second inner shaft 220, and a second balloon 222. A proximal end of second balloon 222 is mounted to a distal portion of second outer shaft 218 at a second proximal junction 224. A distal end of second balloon 222 is mounted to a distal portion of second inner shaft 220 at a second distal junction 226. A second inflation lumen 225 extends between second outer shaft 218 and second inner shaft 220, and is in communication with an interior of second balloon 222. A first guide wire lumen 228 extends through second inner shaft 220.

First and second inflation lumens 215, 225 may be conventional, and extend from a proximal portion of catheter 200 outside the patient, which is not pictured. First and second inflation lumens 215, 225 are in fluid communication with the interiors of first balloon 210 and second balloon 222. Thus, first and second inflation lumens 215, 225 are used to supply pressurized inflation fluid to first balloon 210 and second balloon 222 when it is desired to inflate the balloons. Inflation lumens 215, 225 are also used to drain inflation fluid from first balloon 210 and second balloon 222 when it is desired to deflate the balloons.

Although first and second guide wire lumens 216, 228 are shown passing through the interior of first and second balloons 210, 222, they need not. For example, the lumens may be affixed to the exterior of the balloon, or the balloon may be formed with a plurality of folds through which the guide wire passes. Alternatively, the guide wire may pass through the folds of the balloon, as noted above with respect to the catheter 100. First and second guide wire lumens 216, 228 may begin and terminate generally at any point along first and second catheter branches 202, 204, but preferably they extend distally of first and second balloons 210, 222, respectively.

Distal portion 209 of first inner shaft 208 and distal portion 221 of second inner shaft 220 are bonded to each other at an adhesive bond 230, as shown in FIG. 11. Distal portions 209 and 221 are bonded to each other during delivery to the bifurcation site such that first and second catheter branches 202, 204 are coupled together during delivery, as described in more detail below. Although several figures, such as FIG. 10, show distal portions 209, 221 separate by what appears to be a significant distance such that distal portions 209, 221 may need to be bent to be adhesively bonded, one of ordinary skill in the art would recognize that distal portions 209, 221 may be very close to each other, such as illustrated in FIG. 11.

Once at the lesion site, a force separates distal portion 209 from distal portion 221, thereby freeing first and second catheter branches 202, 204 from each other. The method for delivering the force can be those described above with respect to catheter 100.

With reference to FIGS. 12-16, the manner of practicing this embodiment will now be discussed. A first guide wire 240 is in place in the body lumen. A proximal end of first guide wire 240 is threaded into a distal opening 238 of distal portion 209 of first inner shaft 208. First catheter branch 202 is threaded over first guide wire 240 while distal portion 221 of second inner shaft 220 is adhesively bonded to distal portion 209 of first inner shaft 208, thereby coupling the distal portions of first and second catheter branches 202, 204 together, as shown in FIG. 12.

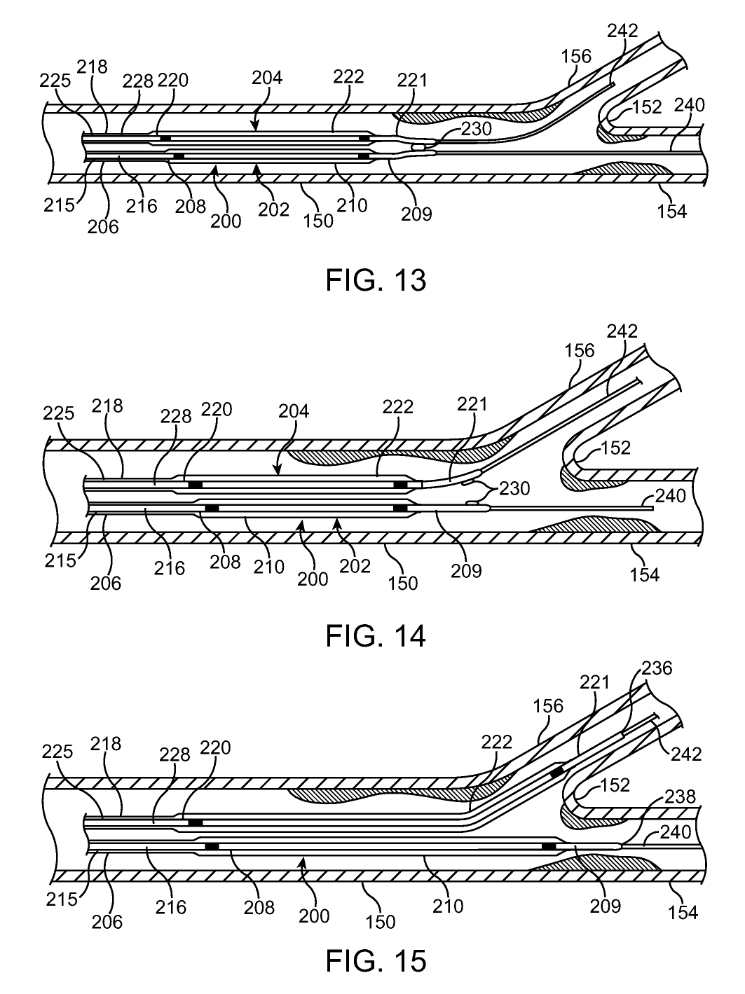

Catheter 200 is thus threaded over first guide wire 240 and tracked to a position at or near a bifurcation 152 of a vessel 150, as depicted in FIG. 12. A second guide wire 242 may be pre-installed through second guide wire lumen 228 such that second guide wire 242 is advanced with catheter 200 as catheter 200 is advanced to the bifurcation site 152. Alternatively, second guide wire 242 may be inserted in second guide wire lumen 228 after catheter 200 has reached the bifurcation site 152. Once catheter 200 is near bifurcation 152, second guide wire 242 is advanced through a distal opening 236 in distal portion 221 of second inner shaft 221 and into second branch vessel 156, as illustrated in FIG. 13.

Catheter 200 is then advanced over first and second guide wires 240, 242. As catheter 200 approaches bifurcation 152, first catheter branch 202, which is tracking over first guide wire 240, tracks towards first branch vessel 154. Meanwhile, second catheter branch 204, which is tracking over second guide wire 242, tracks towards second branch vessel. The divergent paths of first catheter branch 202 and second catheter branch 204 breaks adhesive bond 230 such that first and second catheter branches 202, 204 are separated from each other, as shown in FIG. 14.

With adhesive bond 230 broken such that first and second catheter branches 202, 204 are separated, first and second catheter branches 202, 204 can be positioned independently of one another such that first and second balloons 210, 222 may be positioned independently of each other. First and second catheter branches 202, 204 are advanced into first and second branch vessels 154, 156, respectively, as illustrated in FIG. 15.

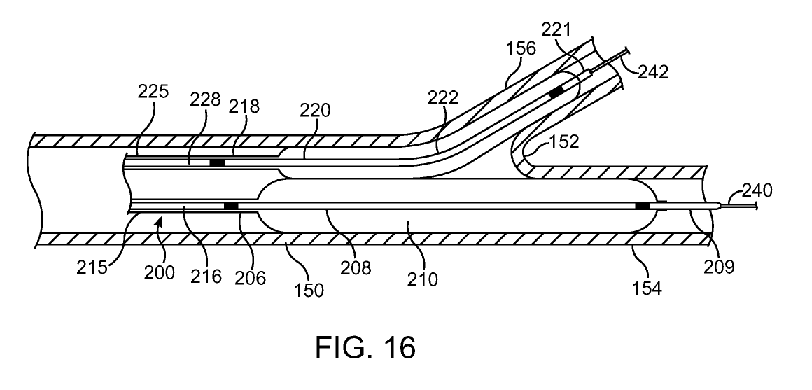

Once the entire assembly is properly positioned, pressurized fluid is supplied to first and second balloons 210, 222 through first and second inflation lumens 215, 225, as shown in FIG. 16. After first balloon 210 and second balloon 222 have been inflated as described above, first balloon 210 and second balloon 222 are deflated by draining the inflation fluid via first and second inflation lumens 215, 225. This allows the balloons to collapse in preparation for withdrawal of the assembly from vessel 150.

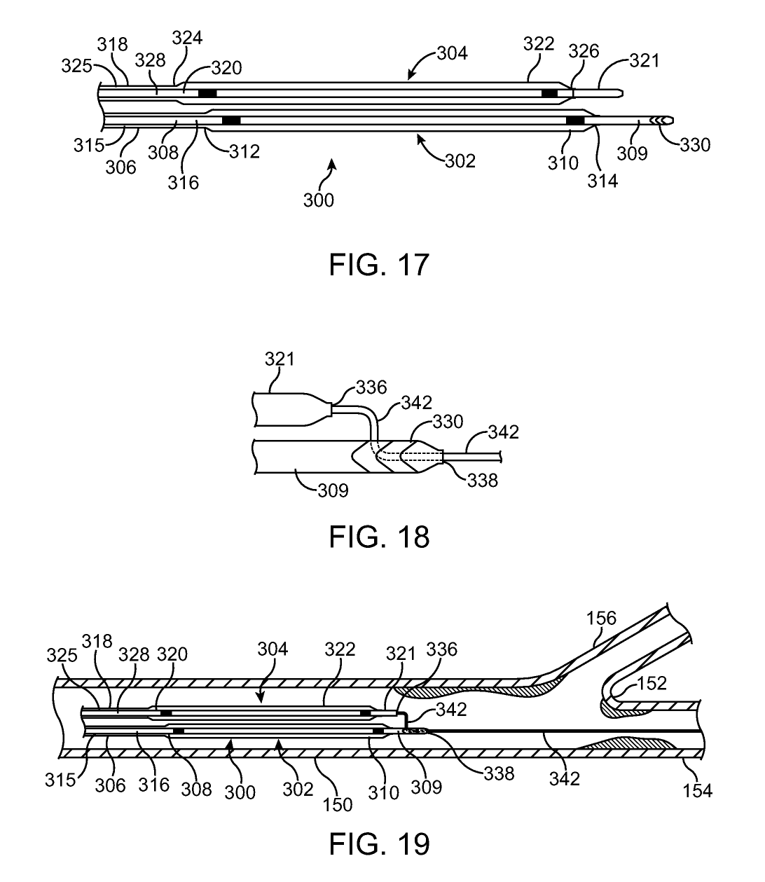

FIGS. 17-25 depict another embodiment of a bifurcated catheter 300 in accordance with the present invention. Catheter 300 includes a first catheter branch 302 and a second catheter branch 304. First catheter branch 302 includes a first outer shaft 306, a first inner shaft 308, and a first balloon 310. A first inflation lumen 315 extends between first outer shaft 306 and first inner shaft 308, and is in communication with an interior of first balloon 310. A first guide wire lumen 316 extends through first inner shaft 308. Similarly, second catheter branch 304 includes a second outer shaft 318, a second inner shaft 320, and a second balloon 322. A second inflation lumen 325 extends between second outer shaft 318 and second inner shaft 320, and is in communication with an interior of second balloon 322. A first guide wire lumen 328 extends through second inner shaft 320.

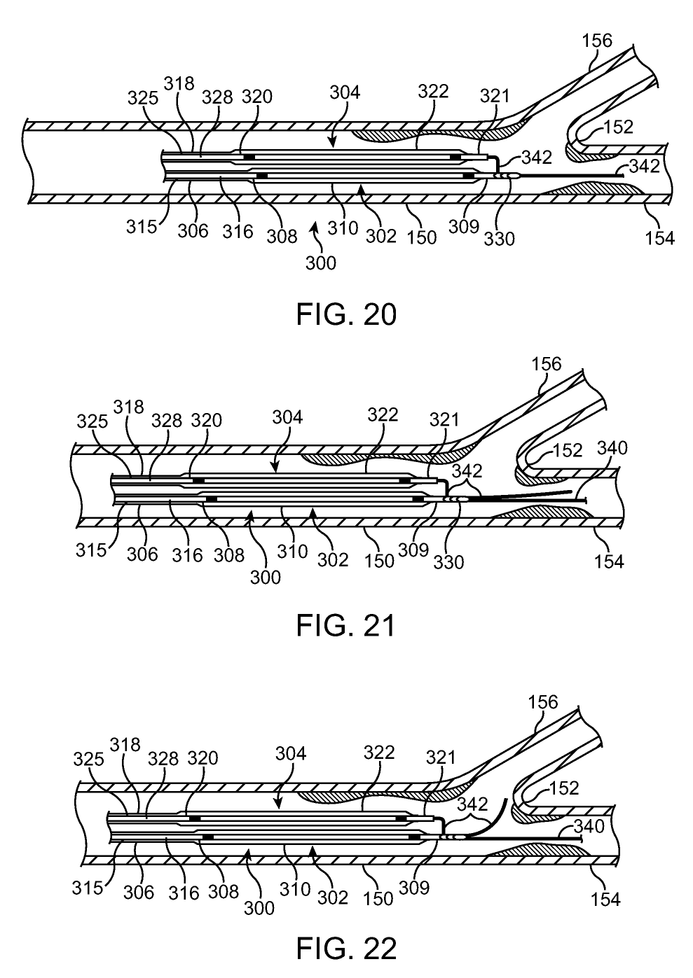

A distal portion 309 of first inner shaft 308 includes a spiral 330, as best seen in FIG. 18. A proximal end of a first guide wire 342 is inserted into a distal opening 338 of first inner shaft 308. First guide wire 342 exits distal portion 309 of first inner shaft 308 through spiral 330 and is insert into a distal opening 338 of second inner shaft 320. First and second catheter branches 302, 304 are thus threaded over first guide wire 342 while first guide wire 342 is couples distal portions 309, 321 of first and second catheter branches 302, 304 together. Catheter 300 is thus threaded over first guide wire 342 and tracked to a position at or near bifurcation 152 of vessel 150, as illustrated in FIGS. 19 and 20.

A second guide wire 340 may be pre-installed through first guide wire lumen 316 such that second guide wire 340 is advanced with catheter 300 as catheter 300 is advanced to the bifurcation site 152. Alternatively, second guide wire 340 may be inserted in first guide wire lumen 316 after catheter 300 is near the bifurcation 152. Once catheter 300 is near bifurcation 152, second guide wire 340 is advanced through a distal opening 338 in distal portion 309 of first inner shaft 309 and into first branch vessel 154, as illustrated in FIG. 21.

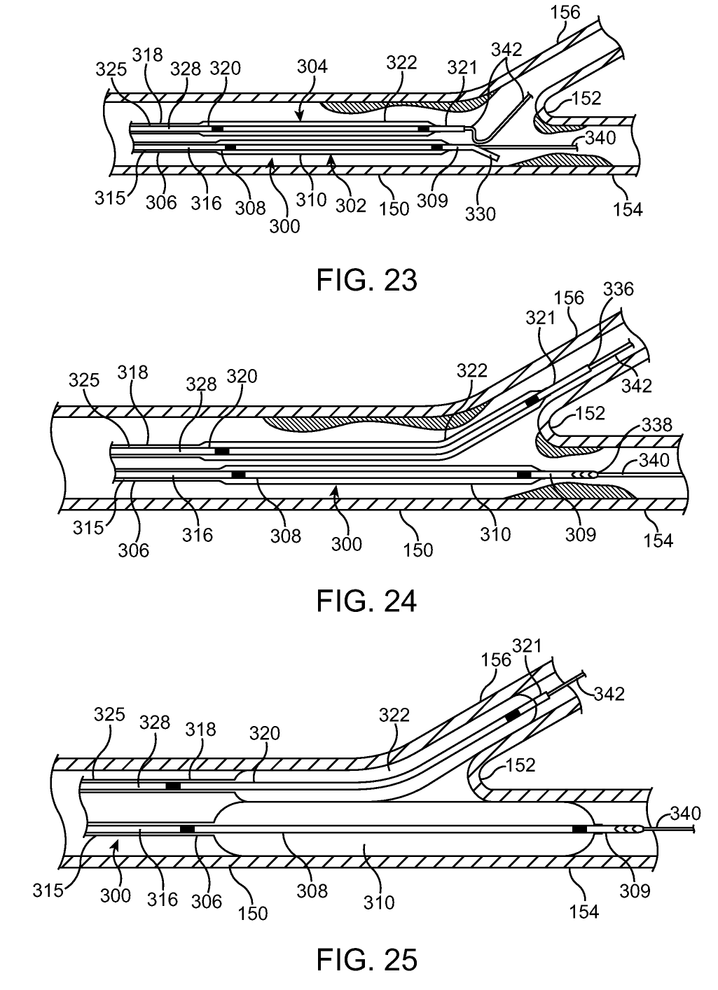

First guide wire 342 is then backed out of first branch vessel 154 and advanced towards second branch vessel 156, as illustrated in FIG. 22. As first guide wire 342 is advanced into second branch vessel 156, first guide wire 342 pulls through spiral 330 of distal portion 309 of first inner shaft 308, as illustrated in FIG. 23. First guide wire 342 is thus freed from distal portion 309 of first inner shaft 308, thereby freeing first and second catheter branches 302, 304 from each other. Spiral 330 is sufficiently pliant that it wraps itself back around second guide wire 340, as illustrated in FIG. 24.

First guide wire is further advanced into second catheter branch 156 and first and second catheter branches 302, 304 are advanced into first and second branch vessels 154, 156, respectively, as illustrated in FIG. 24. Once the entire assembly is properly positioned, pressurized fluid is supplied to first and second balloons 310, 322 through first and second inflation lumens 315, 325, as illustrated in FIG. 25. After first balloon 310 and second balloon 322 have been inflated as described above, first balloon 310 and second balloon 322 are deflated by draining the inflation fluid via first and second inflation lumens 315, 325. This allows the balloons to collapse in preparation for withdrawal of the assembly from vessel 150.

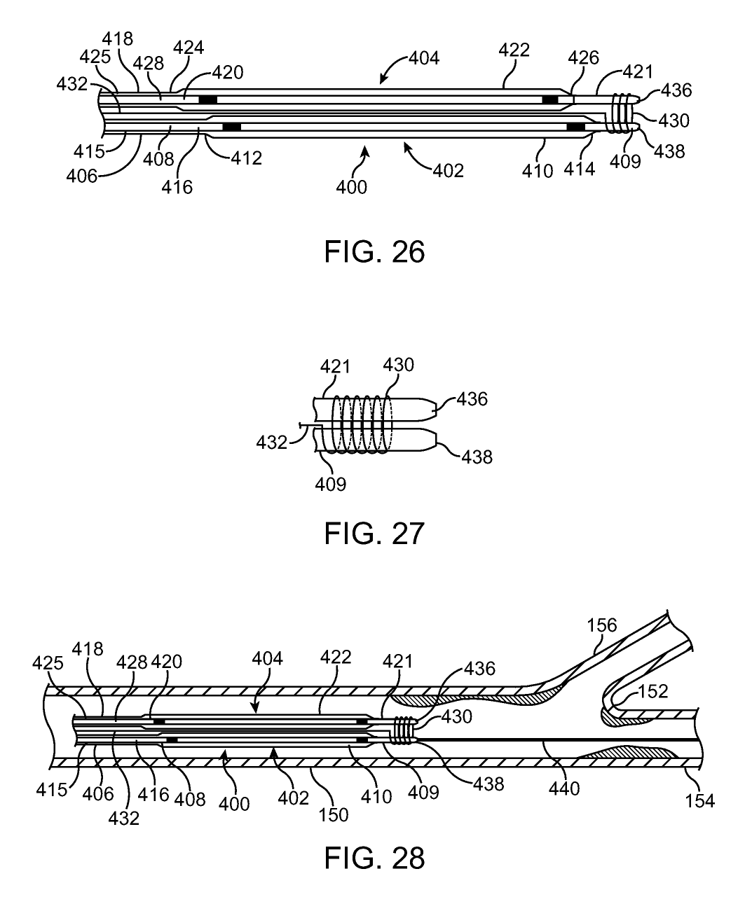

Another embodiment of a bifurcated catheter 400 is described with respect to FIGS. 26-32. Catheter 400 includes a first catheter branch 402 and a second catheter branch 404. First catheter branch 402 includes a first outer shaft 406, a first inner shaft 408, and a first balloon 410. A proximal end of first balloon 410 is mounted to a distal portion of first outer shaft 406 at a first proximal junction 412. A distal end of first balloon 410 is mounted to a distal portion of first inner shaft 108 at a first distal junction 414. A first inflation lumen 415 extends between first outer shaft 406 and first inner shaft 408, and is in communication with an interior of first balloon 410. A first guide wire lumen 416 extends through first inner shaft 408.

Similarly, second catheter branch 404 includes a second outer shaft 418, a second inner shaft 420, and a second balloon 422. A proximal end of second balloon 422 is mounted to a distal portion of second outer shaft 218 at a second proximal junction 424. A distal end of second balloon 422 is mounted to a distal portion of second inner shaft 420 at a second distal junction 426. A second inflation lumen 425 extends between second outer shaft 418 and second inner shaft 420, and is in communication with an interior of second balloon 422. A first guide wire lumen 428 extends through second inner shaft 420.

First and second inflation lumens 415, 425 may be conventional, and extend from a proximal portion of catheter 400 outside the patient, which is not pictured. First and second inflation lumens 415, 425 are in fluid communication with the interiors of first balloon 410 and second balloon 422. Thus, first and second inflation lumens 415, 425 are used to supply pressurized inflation fluid to first balloon 410 and second balloon 422 when it is desired to inflate the balloons. Inflation lumens 415, 425 are also used to drain inflation fluid from first balloon 410 and second balloon 422 when it is desired to deflate the balloons.

Although first and second guide wire lumens 416, 428 are shown passing through the interior of first and second balloons 410, 422, they need not. For example, the lumens may be affixed to the exterior of the balloon, or the balloon may be formed with a plurality of folds through which the guide wire passes. Alternatively, the guide wire may pass through the folds of the balloon, as noted above with respect to the catheter 100. First and second guide wire lumens 416, 428 may begin and terminate generally at any point along first and second catheter branches 202, 204, but preferably they extend distally of first and second balloons 410, 422, respectively.

Distal portion 409 of first inner shaft 408 and distal portion 421 of second inner shaft 420 are coupled to each by a coil 430, as illustrated in FIG. 27. Coil 430 is wrapped around distal portion 409 and 421 and includes a pull string 432 which extends proximally to a location outside of the patient's body. Coil 430 is wrapped around distal portions 409 and 421 during delivery of catheter 400 to the bifurcation site such that first and second catheter branches 402, 404 are coupled together during delivery, as described in more detail below. Coil 430 may be made of polymer or wire strands, or any other suitable biocompatible material, as would be understood by one of ordinary skill in the art.

With reference to FIGS. 28-32, the manner of practicing this embodiment will now be discussed. A first guide wire 440 is in place in the body lumen. A proximal end of first guide wire 440 is threaded into a distal opening 438 of distal portion 409 of first inner shaft 408. First catheter branch 402 is threaded over first guide wire 440 while distal portion 421 of second inner shaft 420 is coupled to distal portion 409 of first inner shaft 408 via coil 430, thereby coupling the distal portions of first and second catheter branches 402, 404 together, as illustrated in FIG. 28.

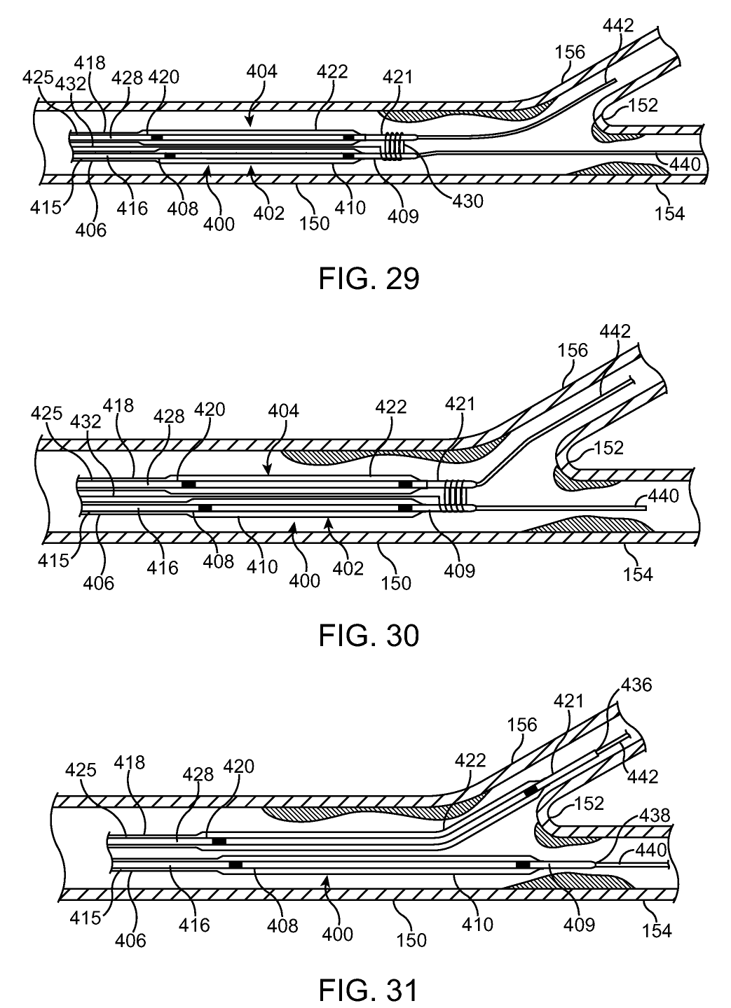

Catheter 400 is thus threaded over first guide wire 440 and tracked to a position at or near a bifurcation 152 of a vessel 150, as depicted in FIG. 28. A second guide wire 442 may be pre-installed through second guide wire lumen 428 such that second guide wire 442 is advanced with catheter 400 as catheter 400 is advanced to the bifurcation site 152. Alternatively, second guide wire 442 may be inserted in a proximal opening of second guide wire lumen 428 after catheter 400 has reached the bifurcation site 152. Once catheter 400 is near bifurcation 152, second guide wire 442 is advanced through a distal opening 436 in distal portion 421 of second inner shaft 4221 and into second branch vessel 156, as illustrated in FIG. 29.

Pull wire 432 may then be pulled such that coil 430 unwinds, thereby freeing distal portions 409 and 421 from each other, as illustrated in FIG. 30. With distal portions 409, 421 freed from each other due to the unwinding of coil 430, first and second catheter branches 402, 404 can be positioned independently of one another such that first and second balloons 410, 422 may be positioned independently of each other. First and second catheter branches 202, 204 are advanced into first and second branch vessels 154, 156, respectively, as illustrated in FIG. 31.

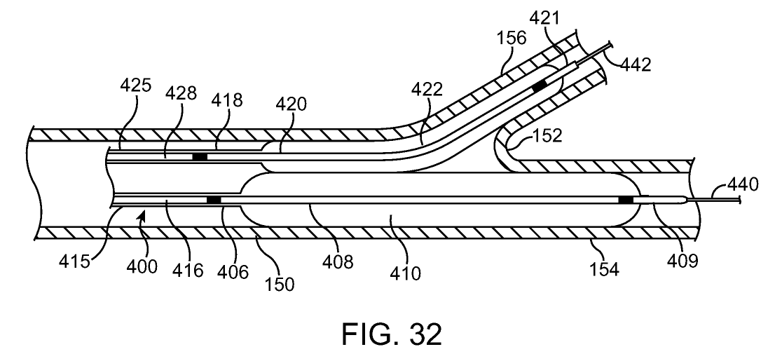

Once the entire assembly is properly positioned, pressurized fluid is supplied to first and second balloons 410, 422 through first and second inflation lumens 415, 425, as shown in FIG. 32. After first balloon 410 and second balloon 422 have been inflated as described above, first balloon 410 and second balloon 422 are deflated by draining the inflation fluid via first and second inflation lumens 415, 425. This allows the balloons to collapse in preparation for withdrawal of the assembly from vessel 150.

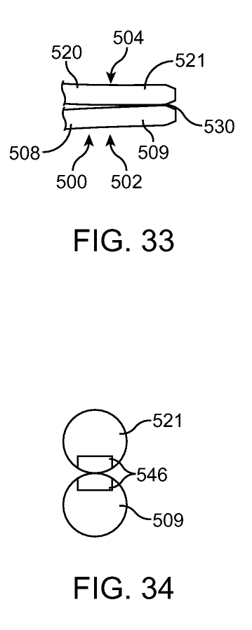

FIGS. 33 and 34 illustrate another embodiment of a catheter 500 in accordance with the present invention. Catheter 500 is similar to catheter 200 described with respect to FIGS. 10-16 in that a distal portion 509 of a first inner shaft 508 of a first catheter branch 502 is bonded to a distal portion 521 of a second inner shaft 520 of a second catheter branch 504. However, instead of an adhesive bond, as described with respect to catheter 200, distal portions 509 and 521 are bonded together through chemical and mechanical bonding. During manufacture of catheter 500, a heated clamp 546 is inserted into distal portions 509, 521, as illustrated in FIG. 34. Heated clamp 546 applies heat and pressure to walls of distal portions 509, 521 adjacent to each other. Such heat and pressure causes the material of distal portions 509, 521 to bond. Catheter 500 is delivered to the site of the lesion in the same manner as described above with respect to catheter 200. First and second catheter branches 502, 504 are separated from each other near the bifurcation in the same manner as described with respect to FIGS. 13 and 14.

The various components of the catheters of this invention can be made of the same materials that are conventionally used for generally corresponding components of known catheters. Thus, for example, the various lumens can be made of materials such as polyethylene, polyethylene terephthalate, polyurethanes, polyesters, polyamides and copolymers thereof.

As another example, at least part of the outer or inner shafts may be stainless steel, polyimide or the like. A polyimide hyptotube or similar material may encase the proximal shaft of the catheter. A sufficiently rigid material may prevent the twisting of the catheter and potential distortion of the lumens and guide wires within the catheter in the event a torque is applied to the catheter during positioning of the device.

The material of the balloons may be polyethylene, polyethylene terephthalate, nylon, polyamides, latex rubber, or other polymer. Guide wires can also be of any conventional construction and material, including solid or braided stainless steel. Hence, the term “wire” is used for these elements only as a matter of convenience, and that the material may not necessarily be wire.

The dimensions (e.g., the lengths, diameters, thicknesses, etc.) of various components of the catheters of this invention may be similar to the dimensions that are conventionally used for generally corresponding components of known catheters.

It would be understood by those of ordinary skill in the art that while the embodiments of the present invention discussed above are described with respect to a dual-lumen catheter including an outer shaft and an inner shaft, several different types of catheters known in the art could be used, for example, rapid exchange type catheters.

While various embodiments of the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

1. An apparatus for treating a bifurcated region of a body lumen, comprising:
a catheter having a first catheter branch and a second catheter branch, wherein the first catheter branch includes a first distal portion and the second catheter branch includes a second distal portion; and
a skive disposed in the first distal portion, wherein the skive is sized and shaped to receive a distal end of the second distal portion.

2. The apparatus of claim 1, further comprising at least one balloon disposed on the first and second catheter branches, wherein the first and second distal portions are disposed distal of the balloon.

3. The apparatus of claim 1, further comprising:
a first balloon disposed on the first catheter branch;
a second balloon disposed on the second catheter branch;
a first inflation lumen disposed within the first catheter branch and in communication with an interior of the first balloon; and
a second inflation lumen disposed within the second catheter branch and in communication with the balloon.

4. The apparatus of claim 1, wherein the first catheter branch includes a first guide wire lumen and the second catheter branch includes a second guide wire lumen.

5. The apparatus of claim 1, wherein the second distal portion is configured to be disposed within the skive during delivery to the bifurcated region, and the

6-20. (canceled)

21. A method for treating a bifurcated region of a body lumen, comprising the steps of:
receiving a catheter having a first catheter branch, a second catheter branch, and at least one balloon disposed on the first and second catheter branches, wherein the first catheter branch includes a first distal portion and the second catheter branch includes a second distal portion, wherein a skive is disposed in the first distal portion, the skive being sized and shaped to receive a distal end of the second distal portion;
inserting the second distal portion into the skive of the first distal portion and securing the second distal portion within the skive;
inserting a first guide wire into the body lumen and advancing the first guide wire to the bifurcated region;
inserting a proximal end of the first guide wire into a first distal opening of the first distal portion and through a second distal opening of the second distal portion;
advancing the catheter over the first guide wire to the bifurcated region;
removing the second distal portion from the skive and removing the first guide wire from the first catheter branch through the skive;
advancing a second guide wire through a guide wire lumen in the first catheter branch and into a first branch of the body lumen;
advancing the first guide wire into a second branch of the body lumen;
advancing the bifurcated catheter over the first and second guide wires such that the first catheter branch advances into the first branch of the body lumen and the second catheter branch advances into the second branch of the body lumen; and
inflating the at least one balloon.

22. The method of claim 21, wherein the first catheter branch includes a first inflation lumen in communication with an interior of the at least one balloon and the second catheter branch includes a second inflation lumen in communication with the at least one balloon.

23. The method of claim 21, wherein the at least one balloon comprises a first balloon disposed on the first catheter branch and a second balloon disposed on the second catheter branch, and wherein the first catheter branch includes a first inflation lumen in communication with an interior of the first balloon and the second catheter branch includes a second inflation lumen in communication with the second balloon.

24-33. (canceled)
